(19) United States
(12) Patent Application Publication
(10) Pub. No.: US 2011/0087137 A1
(43) Pub. Date: Apr. 14, 2011

(54) MOBILE FITNESS AND PERSONAL CALORIC MANAGEMENT SYSTEM

(76) Inventor: Reed Hanoun, Mississauga (CA)

(21) Appl. No.: 12/999,354

(22) PCT Filed: Jun. 11, 2009
(86) PCT No.: PCT/CA09/00828

§ 371 Date: Dec. 16, 2010

Related U.S. Application Data
(60) Provisional application No. 61/061,869, filed on Jun. 16, 2008.

(30) Foreign Application Priority Data
Mar. 11, 2009 (CA) CA2009/000303

(51) Int. Cl. G09B 19/00; A61B 5/11; A61B 5/02

(52) U.S. Cl. 600/587; 434/247; 434/127; 600/508

(57) ABSTRACT

A user's personal biometric information such as age, sex, weight, height as well as the user's lifestyle information, such as daily caloric input, job description, smoker status and physical fitness, is uploaded onto a host computer. Target heart rate, energy and/or caloric consumption levels related to desired fitness and weight loss goals for a particular individual are then selected having regard to fitness levels for an individual of comparable age, and consuming similar calories are then downloaded to a caloric monitoring unit. The caloric monitoring unit is provided for measuring the user's heart rate and dynamic energy and/or caloric expenditure over one to four weeks. The caloric monitoring unit includes a heart rate monitor, a unit accelerometer, a global positioning system (GPS), and an audio and/or video output. The audio/video output is operable to provide information and/or motivational prompts to the user in the event the heart rate, energy expenditure and/or caloric expenditure falls below or exceeds pre-selected target expenditures over a particular time segment of the selected time period having regard to the calories which are consumed. A display provides a continuously updated visual indication of whether or not the use has achieved the pre-selected optimum caloric burn or energy expenditure for that particular time segment. An internal calendar/clock, a processor and/or memory in the caloric monitoring unit compares measured heart rate and energy expenditures for multiple time segments against target levels stored as the user-specific fitness programme tailored to achieve the desired weight loss. The comparison is then used to generate compliance output data to either the user and/or a nutritionist.

### RELATED APPLICATIONS

This application claims the benefit of priority under 35 USC §119(e) to U.S. Provisional patent application Ser. No. 61/061,869 filed 16 Jun. 2008, and entitled “Mobile Fitness Enabling Device”.

### SCOPE OF THE INVENTION

The present invention relates to a mobile fitness and caloric management system, and more particularly a personal caloric management system which is operable to both coach and allow one or more users to continuously monitor both their daily caloric intake, and also caloric expenditure or burn over a 24 hour period or longer, to achieve a more balanced personalized fitness and/or weight loss goal.

### BACKGROUND OF THE INVENTION

Computerized fitness systems used in health clubs are well known. These systems allow a user to input into a health club computer a target weight or physical fitness goal. During in-club exercise workouts, the computer operates to provide the user with feedback as to whether or not pre-selected milestones, such as target heart rates or reps, have in fact been reached.

The inventor's United States patent application publication No. US 2007/0232455 A1 to Hanoun, published 4 Oct. 2007, describes one such computerized physical activity monitoring system. The system described in Hanoun allows individuals to pre-program a desired level of physical fitness as part of a health club facility workout. Using a unique radio frequency identifier (RFID) or chip containing data tag, information is collected from various club exercise machines and workstations which are fitted with sensors related to the user's workout regime. Collected data, such as heart rate information is stored on the tag at each workstation. This information is then downloaded to the health club computer at the end of the exercise workout, to allow the user to extrapolate his or her performance and/or compare it to performance information previously stored in the database or selected target workout intensities. The system described in United States patent application publication No. US 2007/0232455 A1 advantageously provides users with specific information relating to their performance during a selected workout at the health club. Heretofore, however, conventional exercise management systems have failed to allow users to accurately monitor and manage caloric expenditures in light of caloric intake, and which is an important factor in achieving any balanced weight management and overall health.

Other systems have been proposed which allow the user to input estimated caloric consumption. These systems are however, inheritably inaccurate, as they rely on the user's own calculations as to the likely amount of consumed calories. Furthermore, an individual user may not have the necessary expertise to accurately determine the actual calories which are consumed. In addition, such systems typically operate on an honour basis, and may be subject to inadvertent or intentional omissions. As a result, conventional computerized monitoring systems have proven ineffective in correlating the participant's monitored physical activity with his or her caloric input. As such, if a user tends to consume excess and/or unhealthy calories, despite the improved heart rates, the participants may continue to suffer from increased weight gain and an unhealthy lifestyle.

In addition, various nutrition focused weight-loss programs exist which allow users to select meals and food choices by caloric loading, as part of weight management programs. Typically, these programs allow participants to purchase prepared meals which are chosen and portion-sized to provide a predetermined measured caloric intake. The user may therefore select from a number of different types of pre-selected foods or prepared meals which provide a reduced caloric input chosen to achieve a desired weight loss over a given period of time, and which are delivered to the user on enrolment. Nutrition focused weight loss management systems do not, however, provide any feedback as to whether or not the participant is adhering to the selected dietary regime, or otherwise cheating by consuming meals or snacks outside those which are supplied.

### SUMMARY OF THE INVENTION

The present invention seeks to provide a mobile fitness and caloric management system which allows one or more users to monitor and manage both their total daily caloric intake, as well as their total daily caloric expenditure as part of an overall fitness and/or wellness regime.

Another object of the invention is to provide a caloric management system which allows multiple users to pre-select a user specific desired fitness and/or weight level, and to purchase from third parties, prepared meals and/or exercise programs which are tailored to achieve an optimum balance between caloric intake and caloric expenditure to best achieve the desired fitness or weight level.

Another object is to provide a system which allows a user and/or third parties to continuously monitor an individual's daily caloric expenditure, and which provides the user with visual and/or aural feedback, exercise programs and/or motivational prompts, in the event the actual measured caloric expenditures do not meet or exceed a target caloric expenditure for a particular time of day.

Yet another object of the invention is to provide a coaching, programming and monitoring system for ensuring compliance with a pre-selected exercise and/or dietary-programme which is selected to achieve a desired weight loss and improve overall health.

A further object of the invention is to provide a computerized weight management system which allows one or more users to order from third parties pre-selected dietary meals and/or pre-proportioned foods which are tailored to provide a predetermined caloric intake selected to achieve desired weight loss and/or fitness goals. More preferably, the system allows for the monitoring by the user and/or the third party of the user's total daily caloric expenditures to provide an indication of whether or not the user is consuming foods outside the pre-selected program, and/or modifies or adjusts the exercise programmes and/or future meal or food choices, depending on whether or not the user achieves the target caloric expenditure.

In a simplified construction, a personal digital assistant or caloric monitoring unit (CMU) is provided for measuring the user's total caloric expenditure throughout the day. The caloric monitoring unit is designed to be portable, so as to be easily and comfortably worn by the user when not only exercising at a health club, but at substantially all times throughout his or her waking day. The CMU preferably includes one or more of a heart rate monitor, an accelerometer, a global positioning system (GPS), an Mp3 player and an audio and/or video output operable to provide motivational prompts to the user. More preferably, the CMU provides motivational prompts in the event the caloric expenditure does not equal or exceed a pre-selected target optimal caloric expenditure chosen to achieve a desired weight loss or overall fitness level. In a preferred construction, the CMU includes a display which is used to provide a visual display providing an indication of whether or not the user has achieved the pre-selected optimum caloric burn or expenditure over a particular time segment at a particular time of day.

The CMU is provided as part of an overall fitness programme monitoring system and is operable to electronically communicate with a central processing unit (CPU). The CPU may take the form of a personal computer, a computer server housed in a health club, or a remote computer maintained by third parties, and which connects to the CMU via the internet or other suitable electronic connection. The CPU most preferably contains a reference datafile which stores a number of exercise programmes, each tailored to achieve a preferred target heart rate and/or joule or caloric expenditure. Most preferably, the exercise programmes are furthermore correlated to an individual's age, sex, weight and/or other biometric parameters. More preferably, the exercise programmes are furthermore correlated to lifestyle profile parameters which may include without restriction the user's daily caloric intake, factors such as whether or not the user is a smoker and/or has other disabilities or impairments, as well as indications of the user's overall health or fitness (i.e. whether the user is presently extremely fit, moderately fit, morbidly obese, etc.).

A client database is provided for storing both target fitness and/or weight loss data as well as client data which is unique to the individual users. Input client data most preferably includes particulars such as the individual's biometric parameters (i.e. age, sex, weight, etc.) as well as lifestyle information. Upon receiving input client data, the central processing unit is operable to download to the CMU an exercise programme which correlates to the input data, and which is selected to achieve a desired weight loss and/or overall fitness result.

Most preferably the user inputs information as to his or her age, sex, daily weight, as well as fitness and/or weight goals which are to be obtained into a computer (PC) for uploading either wirelessly or via the internet onto the host computer/portal or central processing unit (CPU). Target fitness levels may be related to selected fitness goals, or may represent average fitness or Personal Activity Level for individuals of comparable age, and optionally, those performing a similar job function. Alternately, the user may select unique target fitness levels having regard to desired weight loss and/or weight gain.

More preferably, the CMU is provided with an internal calendar/clock and a processor and/or memory which compares the optimum caloric expenditure for a given time over a pre-selected daily routine (typically the user's waking day over a fifteen to eighteen hour period), against the user's actual measured caloric expenditure for the actual measured time. The CMU may thus be operable either inside and/or outside health club environments to continuously record and monitor the user's “real-world” activities.

In one mode of operation, the system operates automatically, based on understanding the user's body type, age, height, body mass index, and/or with other biometric variables to calculate and predetermine the amount of calories an individual has been consuming on a daily basis to arrive at their current body weight. The approach of calculating calories, also referred to as “trending caloric intake” TCI, does not require the use of any meal planning or food selection in order to measure the number of calories an individual is consuming. This approach simplifies the engagement for users to establish an effective exercise for selected health goals, designed specifically to burn off at least the minimum trending caloric intake in order to maintain a current weight and/or burn off additional calories through exercise and daily activity.

The process of determining trending caloric intake occurs each time the user enters their body weight in the system software. By factoring all of the user biometric variables, and tracking historical physical activity performed by the user while wearing the CMU, the system is operable to estimate exercise benefits on the body.

In a further operational mode, the system is operable to determine the health of one's individual heart without the use of dedicated heart measuring sensors, such as the heart chest straps or ECG type modalities. This is done based on understanding the biometric data for an individual; tracking accurately their health activity using CMU over a period of time; and determining the exertion levels that were required and duration of time that the activity was sustained to yield an energy burn for the muscles in order to sustain such activity. With the forgoing information, it is possible to extrapolate that in order for the body (in particular musculoskeletal system) to sustain the activity, an energy burn would require the cardiovascular system to be able to support a predetermined level of exertion. For each given time period, the higher the activity, the more stress the body experiences, the more demand the heart is going to be under to deliver the required blood flow to the muscles.

On the assumption that the heart is able to deliver blood flow to the musculoskeletal system, it is possible to determine and convert that level of work into a heart health score. Over a measured period of time it is possible to calculate and establish a correlation between the energy burned through physical activity compared to the predicted response and the effort required by the heart.

In addition, by the periodic optional use of a heart belt or heart monitoring sensor, (or through some other modality used to establish heart rate) the user's heart rate can be used as a baseline or benchmark to validate calculated algorithms and the predictions used to calculate heart function, and reset the assumptions for subsequent calculations.

Similarly to calculating trending caloric intake, the CMU may be used to calculate and track, on average based on a defined timeline, the trending caloric output as well. By accurately tracking body movement on a selected time (i.e. minute by minute) basis over an extended period of time, data can be tabulated. The tabulated output can be used to inform the user of the resulting comparison of the user's energy or caloric output, the trending caloric input, predicting weight loss or weight gain.

Although not essential, in an alternate embodiment, a reference database stores as lifestyle profile parameters, caloric information related to a number of pre-selected pre-proportioned meals and/or food choices. The client database allows the user to pre-select and order a number of such pre-selected meals and/or foods (via a home computer or internet login, etc.) for purchase and delivery to the user. In this manner the individual exercise programme which is downloaded to the user's CMU may be adjusted to compensate for the user's anticipated caloric intake for a pre-selected period of time. If the information uploaded from the CMU indicates that the target fitness levels or fitness goals are not being met, the CPU may then adjust the meal choices which are available and/or which are shipped to the user, to provide a reduced number of calories and/or reflect either the compliance and/or non-compliance with the selected exercise programme.

In another construction, a caloric monitoring unit (CMU) is provided for measuring the user's caloric consumption and/or dynamic energy and/or caloric expenditure over an extended period of time. Preferably the time period extends over the bulk of at least one waking day, and most preferably over a dietary programme lasting at least one to four weeks. The CMU includes internal memory, one or more of a heart rate monitor, a unit accelerometer and a global positioning system (GPS). Optionally the CMU may also be provided with a digital audio and/or video Mp3 player and an audio and/or video output. The audio/video output is preferably operable to provide information and/or motivational prompts to the user in the event the heart rate, energy expenditure and/or caloric expenditure falls below or exceeds pre-selected target expenditures which are correlated to a specific pre-selected meal plan over a particular time segment of the selected time period. In a preferred construction, the CMU includes a display to provide periodically an updated visual indication of whether or not the user has achieved the pre-selected caloric burn or energy expenditure having regard to caloric input for that particular time segment. The CMU is preferably provided with wireless communication capability, allowing for its interface with a health club computer system during an in-club workout session, so as to allow the user to upload and download heart rate, dynamic energy and/or caloric expenditure data and/or programming for a variety of in-club exercise activities, such as a selected physical fitness regime, staffed or unstaffed aerobic classes and the like. The health club computer system may be operable to provide for the downloading of heart rate and/or joule expenditure data directly to the CMU, or in an operable configuration may connect electronically directly to the CPU to update the fitness profile for the individual user in the stored client database.

Accordingly, in one aspect the present invention resides in a fitness monitoring and coaching system for ensuring a user's compliance with a pre-selected fitness programme over a selected time period, the system comprising, a data management system including, a reference database for storing datafiles providing preferred target heart rate and joule expenditure standards for a plurality of exercise programmes, said exercise programmes correlated to a user's biometric parameter and a lifestyle profile parameter, said biometric parameters comprising at least one of said user's age and weight, a client database for storing input client data indicative of said user's biometric parameters and a lifestyle profile information for said user, a processing unit operable to output as said pre-selected fitness programme a selected one of said exercising programmes correlated to said input client data, a wearable monitoring device for communicating with said data management system and being operable to measure said user's caloric and/or joule expenditure over the selected time period, the monitoring device including, an internal clock, a display, memory for storing said pre-selected exercise programme as downloaded user specific heart rate and joule expenditure standards over the selected time period, an output for outputting heart rate data singles indicative of said user's actual measured or estimated heart rate over the selected time, an accelerometer for monitoring and outputting joule data signals indicative of at least one of movement by said user; an internal processor for comparing said heart rate data signals and said joule data signals to said user specific heart rate and joule expenditure standards over said selected time period, and outputting a compliance output value which is dependent on the comparison, and wherein the wearable monitoring device is operable to provide on said display in substantially real time a visual representation of said compliance output value, suggestive of a modification to the user's heart rate and/or joule expenditure.

In another aspect, the present invention resides in an monitoring system for monitoring and validating compliance with a pre-selected fitness programme by a user, the system including, a data management system including, a reference database for storing a plurality of datafiles providing preferred target heart rate and caloric and/or joule expenditure standards for a plurality of exercise and nutrition programmes correlated to a selected target weight and fitness profile, and at least one pre-selected biometric parameter selected from a user's age, and weight, a client database for storing client data indicative of biometric parameters of said user, a processing unit operable to output as said pre-selected fitness programme a selected one of said exercise and nutrition programmes which correlates at least in part to at least one of said user's weight and fitness profile, and said pre-selected biometric parameters, a wearable monitoring device for communicating with said data management system and being operable to measure said user's total daily caloric expenditure, the monitoring device including, an internal clock, memory for storing said selected wellness and rehabilitation physiotherapy programme as time dependent user specific heart rate and caloric and/or joule expenditure standards for a pre-selected time period, a heart rate monitoring sensor for monitoring and outputting heart rate data signals indicative of said user's heart rate, and an accelerometer for monitoring and outputting caloric and/or joule data signals indicative of at least one of movement by said user and physical force on at least part of said user's body, a processor for receiving said heart rate data signals and said caloric and/or joule data signals as input values and validating said input values to said user specific heart rate and caloric and/or joule expenditure standards over said pre-selected time period, and outputting a compliance output data indicative of any difference between said input values and said user specific heart rate and caloric and/or joule expenditure standards, a display for providing said user with a visual representation of said compliance output data.

In yet a further aspect, the present invention resides in a method of using a wearable monitoring device for validating a user's compliance with an exercise programme, the wearable monitoring device including an output for outputting heart rate data signals indicative of said user's actual measured or estimated heart rate, and an accelerometer for monitoring and outputting caloric and/or joule data signals indicative of at least one of movement by said user and physical force on at least part of said user's body, the method comprising, providing a reference database storing a plurality of datafiles for preferred target heart rate and caloric and/or joule expenditure standards for a plurality of fitness programmes, said fitness programmes correlated to pre-selected biometric parameters and target weight and/or fitness levels, said biometric parameters comprising at least said user's age and weight, providing a client database storing input client data indicative of a user's age and weight, downloading to the wearable monitoring device as said exercise programme a selected one of said fitness programme correlated to the user's input client data, validating as input values said heart rate data signals and said caloric and/or joule data signals to said user specific heart rate and caloric and/or joule expenditure over a pre-selected time period, and outputting a compliance output data indicative of any difference between said input values and said user specific heart rate and caloric and/or joule expenditure.

### BRIEF DESCRIPTION OF THE DRAWINGS

Reference may now be had to the following detailed description, taken together with the accompanying drawings in which:

FIG. 1 shows schematically a caloric management system in accordance with a preferred embodiment of the invention;

FIG. 2 shows schematically the communication of the caloric monitoring unit and central processing unit of the system shown in FIG. 1;

FIG. 3 illustrates schematically the caloric monitoring unit (CMU) used in the system of FIG. 1;

FIG. 4 shows a perspective view of the personal digital caloric monitoring unit (CMU) for use in the system of FIG. 1;

FIG. 5 illustrates schematically the operation of the CMU of FIG. 2 as part of a circuit training workout in a health club environment; and

FIG. 6 illustrates schematically the operation of the CMU as part of a group fitness workout in a health club environment.

### DETAILED DESCRIPTION OF THE PREFERRED EMBODIMENTS

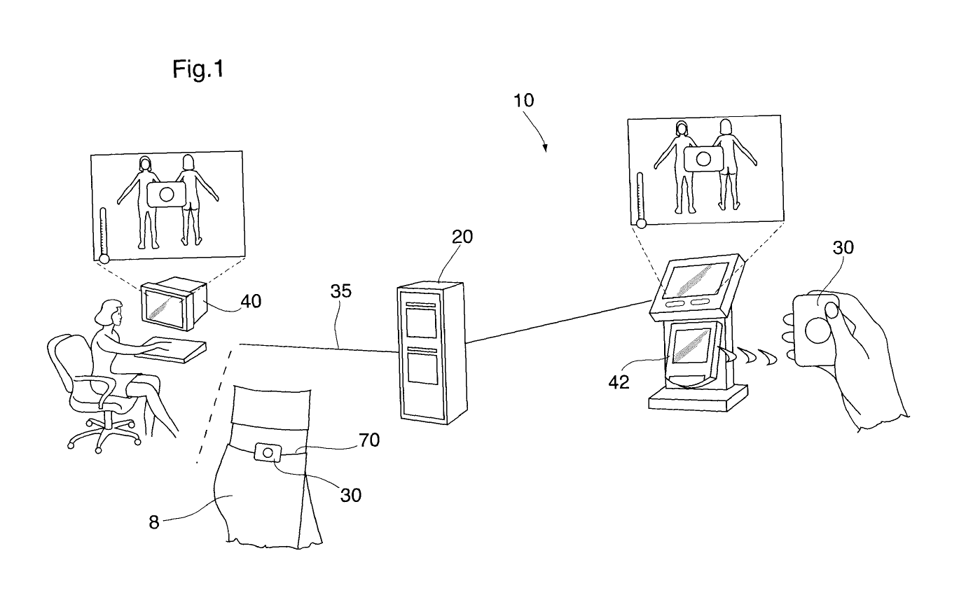

Reference is made to FIG. 1 which schematically illustrates a mobile fitness and personal caloric management system 10 in accordance with a preferred embodiment of the invention. As will be described, the system 10 is most preferably used in the management and monitoring of a user's 8 compliance with a pre-selected nutrition and exercise programme. As will be described, the system 10 provides an advantage over conventional fitness monitoring systems in that unlike existing systems, which focus on in-health club activity over a 45 minute to 1 hour workout, the monitoring system 10 provides for both the monitoring and prompting to maintain compliance with a pre-selected nutrition and exercise regime over an entire waking day for periods of up to weeks or even months. The selected exercise and dietary programmes are chosen having regard to the individual-specific weight loss and fitness needs of the user 8. In use, the present system 10 is operable to provide periodic or real-time visual and aural behavioural modification guides and/or prompts to the user 8 throughout the day.

The system 10 includes a central processing unit (CPU) 20, and at least one, and preferably a number of portable and wearable caloric monitoring units (CMU) 30 operable to be concurrently worn by different multiple users 8 as part of the system operation. Each CMU 30 is configured to download and upload data from the central processing unit (CPU) 20 via a personal computer 40 or a dedicated health club kiosk 42. The CMU 30 has an overall size and configuration selected to be easily carried and worn by the user 8 during both exercise workouts, as well as throughout the daily waking routine, without significantly encumbering daily activities.

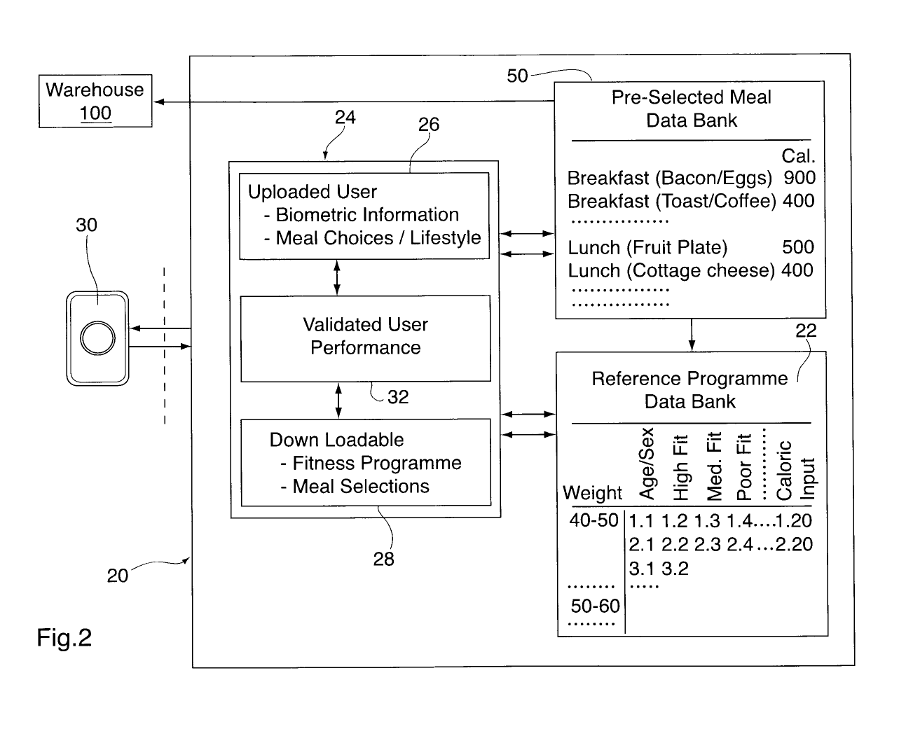

The central processing unit 20 is most preferably housed as part of a mainframe computer housed either at a centralized health club facility, or nutrition management administrator. FIG. 2 shows best the central processing unit 20 as including a reference programme databank 22, and a pre-selected meal databank 50. The reference programme databank 22 contains a series of exercise or fitness modules which are correlated to various biometric profile factors such as a user's weight, age, sex, overall fitness (high fitness, low fitness, poor fitness, etc.). Each of the exercise programmes constitute at least an entire day and preferably a whole week exercise programme which is selected to achieve preferred heart rate and expenditure standards selected to obtained a desired target weight loss and/or fitness level. Most preferably, the exercise programmes are furthermore correlated to the user's anticipated daily caloric input, so as to balance both caloric expenditures with the calories which are consumed by the user 8. The reference programme databank is electronically connected to the administrator meal selection databank 50.

The meal selection databank 50 contains caloric and nutritional information with respect to a number of pre-selected and pre-proportioned meals and foods which are classified by meal type (i.e. snack, breakfast, lunch, dinner), food type (i.e. beef, vegetarian, seafood, etc.) and caloric weighting. Although not essential, the pre-selected meals co-relate to actual pre-packaged prepared meals which are available for direct purchase and shipping to the user 8 as part of an overall weight loss programme. The meal selection databank file 50 may be housed on the CPU 20 itself, or alternately stored at a remote location and linked to the CPU 20 electronically via a local or global computer network connection. Also on the central processing unit 20 is the user's 8 specific client datafile 24. The client datafile 24 includes a client database 26 which stores input client data related to the specific biometric information of the user, as well as lifestyle profile information, and which is either input directly or more preferably uploaded from the user's home PC 40. The input client data preferably contains information specific to the individual user 8 and allows the user 8 to choose a desired fitness goal, such as a target health wellness profile and weight to be achieved over a pre-selected period of time. The pre-selected fitness goal is stored by the CPU 20 as uploaded profile datafile 26 which is unique to the user 8. The user biometric information most typically consists of the user's age, sex and weight. User input lifestyle information may, for example, include an indication of whether or not the user 8 is a smoker or non-smoker, an indication of the user's 8 overall fitness level (i.e. highly fit, moderately fit, morbidly obese, etc.), an indication of any disability of the user, and most preferably a selection of the user's 8 preferred meal choices chosen from the meal databank 50.

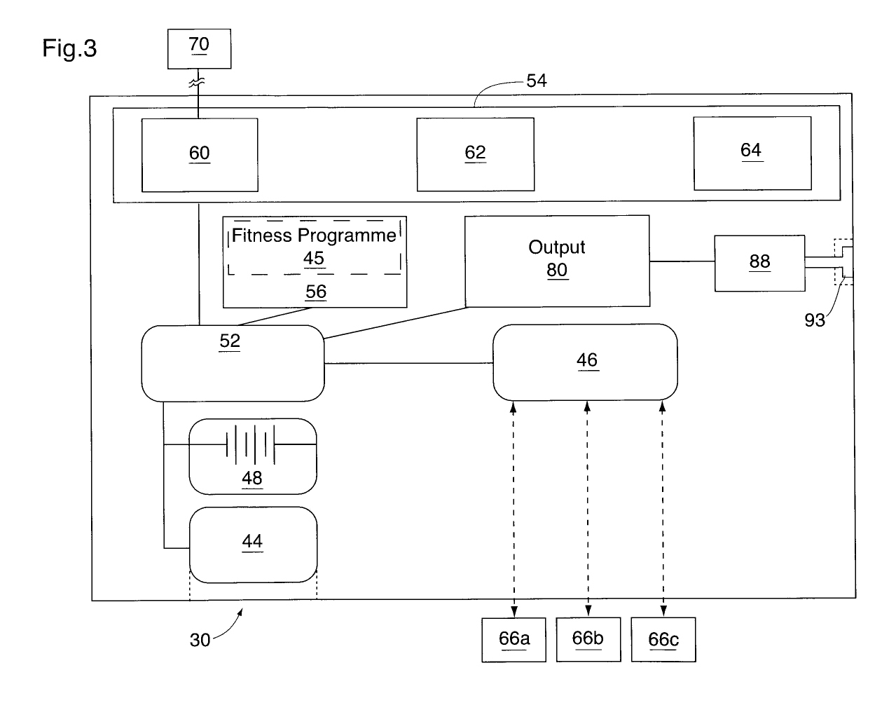

As shown best in FIG. 3, the data file 24 also includes memory 28 for storing specific data including data relating to the pre-selected meals shipped to the user 8 and caloric input, as well as downloaded fitness programmes which are selected having regard to the uploaded user specific information contained in the datafile 24. Information related to the validation of the user's performance and/or adherence with the downloaded fitness programme is furthermore stored as validation record 32 within the client data file 24.

Optionally, other types of lifestyle parameters 26 may also be included as part of the uploaded user data file 26. These may include particulars as to the type of the user's profession and indications of the user's leisure activities. Leisure activity particulars may include whether the individual's daily job activity results in periods of high or low physical activities for a particular time segment during the day (i.e. postman vs. clerical worker) as well as preferred daily caloric indications. The lifestyle parameters may advantageously provide an overall fitness weighting (i.e. active, inactive) or performance index (PI) for the user.

As indicated, the reference programme databank 22 includes a series of individual downloadable pre-selected fitness modules. The fitness modules each include for a selected individual age and gender, pre-selected heart rate and fitness targets or goals, and which may include therein a number of pre-stored downloadable daily exercise programmes. Each of the exercise programmes typically consist of aural instructions and/or video and music which provide a physical workout to the user 8 which are of a duration and/or intensity selected to achieve a predetermined caloric burn. The fitness modules preferably furthermore stipulate preferred user-specific target heart rates, and preferred caloric and energy or joule expenditure standards for both particular types of exercise, as well as for activities throughout the day, and are furthermore linked to the biometric and lifestyle parameters for the user 8 over a pre-selected period of time.

In one possible mode of operation, the fitness module provides the user 8 with instructions to undertake physical activities ranging from stair climbing, to walking or lifting; to more intense cardiovascular workouts which are of a chosen duration and/or intensity when the user 8 is performing daily tasks. The physical activities are selected having regard to the particular time of day, the user's 8 chosen caloric intake (i.e. pre-selected meal/foods) and are chosen to achieve a predetermined heart rate and caloric and/or energy burn. The fitness programmes stored in the databank 22 each provide preferred heart rate and caloric and joule expenditure standards to be achieved throughout individual time segments, such as each half-hour, hour or two-hour period of an average waking day. The caloric expenditure standards preferably account for user activities both when the user 8 is at a health club and performing in-club exercises for a selected time segment (i.e. a particular hour), as well as when undertaking day-to-day activities over a given period.

Ordering software is provided to allow the user 8 to select and order meals via the PC 40, for consumption for a given day, week or month. Typically meals are chosen from the meal databank 50 for the upcoming week or weeks. Following receipt of a meal purchase-order from the PC 40, the CPU 20 transmits the order information to the warehouse 100 for shipping of the pre-prepared foods to the user 8. Meal choices and daily caloric input value of each meal is logged in the CPU 20 in the user's data file 26 for each day over the pre-selected period. The CPU 20 then may adjust the caloric expenditure required for the user 8 to achieve the pre-selected fitness goal having regard to the user's 8 caloric input. In a preferred mode of operation, if the validated user performance data 32 uploaded to the CPU 20 indicates that the user 8 is not achieving the desired caloric expenditure or target heart rate levels stipulated by the downloaded fitness profile, the CPU 20 may adjust the type and/or size of meal selections which are available to the user 8 in the meal databank 50 to reduce the permitted caloric intake. Similarly, if the uploaded validated user performance indicates that the user is exceeding the target joule expenditure and/or heart rates, the CPU 20 may allow the user 8 to choose a wider selection of prepared meals or foods from the pre-selected meal databank 50.

In a more preferred possible construction, the user 8 lifestyle parameters include the user's 8 general class of job category. Job categories for each user 8 are preferably pre-allocated with an average daily caloric/joule expenditure for selected time segments (i.e. each hour/pre-breakfast, mid-morning, lunch hour, etc.) throughout the day. As a result the downloaded fitness programme is operable to provide fitness prompts to the user 8 throughout the working day. In particular, the exercise programme provided by the CPU 20 is most preferably calibrated to increase or decrease desired heart rate and/or physical activity target expenditures in accordance with projected expenditures having regard to equivalent lifestyle parameters, the specific time of day and anticipated activity. For example, during weekday business hours, the downloaded fitness programme adjusts to reflect that the user's 8 ability to provide elevated heart rates and physical activities may be limited by his or her employment duties. During these time segments, the target data operates to provide only moderate aural and/or visual cues to the user 8 to adjust heart rate or activities tailored to both desired rehabilitative exercises and real world expectations.

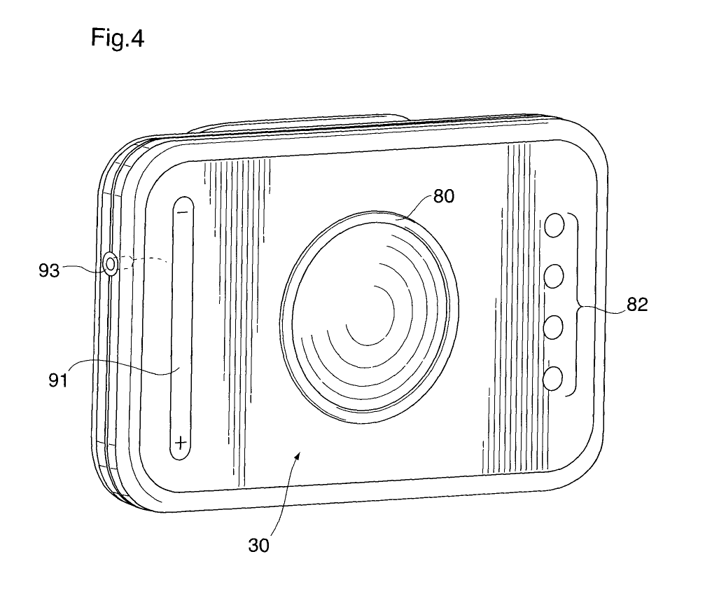

FIGS. 3 and 4 show schematically the caloric monitoring unit 30 in accordance with a preferred embodiment. As will be described, the CMU 30 is operable to communicate with the central processing unit 20 to both download and store a user specific fitness programme, as well as to upload the user's validated performance or compliance data. In a simplified mode of operation, data transfer between the CPU 20 and the CMU 30 is achieved by the uploading and downloading of datafiles via the PC 40 through a communicating network 35 (FIG. 1) such as a conventional home internet connection. The PC 40 may also be used to download fitness progress reports from the CPU 20 to monitor the overall progress of the user 8 in meeting his or her fitness goals.

Data downloading between the CMU 30 and the network 35 is achieved either wirelessly or through a hardwired connection to the PC 40.

FIG. 3 shows best each CMU 30 as having an internal USB connector port 44 for electronically coupling the CMU 30 with the PC 40 so as to facilitate the electronic downloading and uploading of programmes and data. The CMU 30 may also incorporate a Bluetooth™ or other wireless radio module 46 operable to communicate to both the health club kiosk having an RFID reader and/or a house box, such as a wireless router on a PC network. Wireless module communications may be activated through a hall-effect sensor which is operable to detect a magnet housed in the docking station at a health club or home PC 40. Alternatively, the PC 40 could be omitted entirely, with either wireless or direct wired communication occurring between the CPU 20 and CMU 30, or by hardwiring, or direct plug-in.

The CMU 30 has an overall size and weight selected so as to be comfortably worn by the user 8 as a fully portable device. The CMU 30 is therefore preferably provided as a small multi-function mobile fitness monitoring and tracking device. The CMU 30 is adapted to be worn comfortably by the user 8 both during health club exercising, as well as throughout the remainder of the entire waking day, and also most daily tasks. The CMU 30 is operated by way of a rechargeable battery 48 and preferably is approximately the size of a small cell phone or pager. The rechargeable battery 48 may be chargeable through the USB connection port 44 in a conventional manner, as for example during data downloading and uploading from the PC 40, or a wall charging device. A sleep mode and wake-up function are preferably used to conserve power and provide an expected battery life in excess of seven days.

In addition to the battery 48, the CMU 30 is provided internally with an internal calendar/clock processor 52, a performance sensor array 54 and internal portable data storage/memory 56. The sensor array 54 includes a number of different types of sensors used to measure different physical attributes of the user 8 over the course of the pre-selected time segments, such as each hour, throughout the waking day. The sensor array 54 is electronically linked to both the clock/processor 52 and the data storage/memory 56 to allow for the comparison and validation of measured user data, such as heart rate, caloric burn and/or energy burn, against both the input caloric values correlated to the pre-selected target values provided by the fitness programme 45 downloaded from the CPU 20 and stored in the memory 56. In a simplified construction a master output display 80 and operational mode control buttons 82 (FIG. 4), allow the user 8 to select a specific operating mode for the CMU 30, and either upload to the CPU 20 data relating to the validation of the user's 8 heart rate, caloric and/or energy expenditures and/or download exercise programmes for playback. The device memory 56 is used to receive and store the pre-selected exercise programme 45 which is downloaded by the CPU 20 and which is tailored specifically having regard to user's 8 caloric input, the input client data, and the specific user 8 physical fitness or weight loss goal.

The sensor array 54 includes an optional heart rate sensor 60, an internal unit accelerometer 62, and optionally a global positioning sensor (GPS) 64. The CMU 30 may furthermore be operable to communicate with and receive signals from health club equipment sensors 66 via the radio module 46. In this manner the CMU 30 is operable to receive data from sensors 66a, 66b, 66c mounted to health club exercise bikes 102, treadmills 104, rowers 106 and the like.

In one possible construction, the heart rate sensor 60 includes a remote contact sensor pad 70 which is adapted for placement against a pulse point on the user's 8 skin in the monitoring and recording of heart rate data signals which reflect the user's 8 heart rate during CMU 30 operation. Most preferably the sensor 60 is operable to wirelessly receive heart rate pulse data from a wireless chest strap heart rate sensor. Heart rate data is monitored and stored periodically (i.e. every 5 minutes) in the memory 56. More preferably heart rate variability is also calculated and stored as a value. In a preferred mode, the heart rate sensor 60 is used to monitor whether or not a prescribed target heart rate has been achieved for a number of given time segments throughout the entire day. This measured heart rate is then compared against the target heart rate for the corresponding time segment in the downloaded fitness programme 45.

The internal accelerometer 64 is preferably of a 3-axis operational design which is used to measure whole body motion when the CMU 30 is worn on the user 8. By providing a belt mount 70 (FIG. 1), the orientation of the accelerometer 62 with relation to the user's 8 body is advantageously fixed so that the output from each axis is directionally known. In one preferred operational mode, the x-axis is down (giving a +1g signal when stationary). The y-axis is selected forward and the z-axis is inwardly towards the body (i.e. twisting). In general, signals from the accelerometer 62 are continuously analysed together with signals from the GPS sensor 64 and the type of activity determined, with both the activity type and activity dynamic energy stored in memory 56. The data is analyzed in real time, allowing the types and extent of the user's 8 activity to be determined, as for example:
Walking and distance; (Note: Position the device)
Running and distance; (as a body motion and analysis)
Jumping and distance; (device that can be used to determine steps as one of its functions)
Sitting/Standing;
Lying Down;
Rolling;
Passive transportation distance (i.e. car or other transportation journey);
Dynamic energy and activity.
   The accelerometer 62 is thus operable to monitor and output joule data signals which provide an indication of the movement of the user 8. Additionally, the microprocessor 52 provides an analysis algorithm used to filter the accelerometer 62 data and determine the validity of a step. Step count and time is then recorded and saved as time-stamped measured user performance data in the memory 56 periodically (i.e. every 5 minutes). In addition the bounce height of the x-axis is analysed to gain a measure of distance traveled during each step.

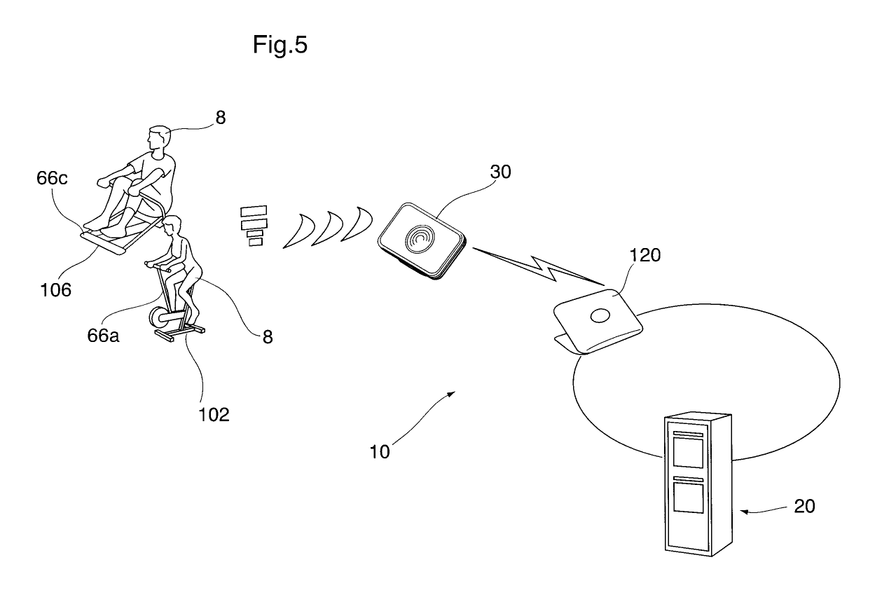

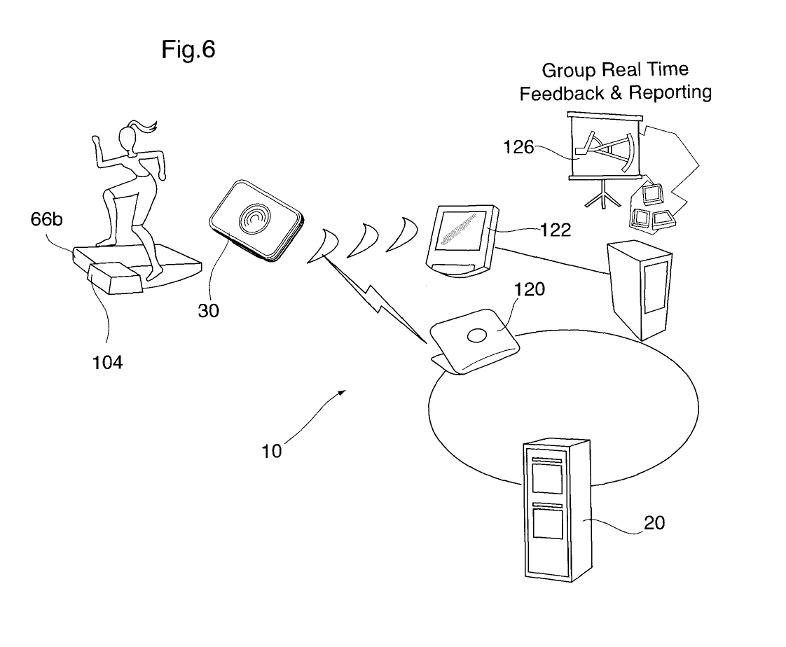

For in health club use, as shown in FIGS. 5 and 6, health club equipment sensors 66a, 66b, 66c provide joule data signals to the processor 52 which are indicative of the user's 8 activation and movement of the specific exercise equipment, such as exercise bikes 102, treadmills 104, rowers 106, and/or the physical force or exertion performed by the user 8 thereon during a health club workout. Sensors 66 are most preferably wirelessly linked to the CMU 30, although hardwired sensors may also be used.

In one possible mode of operation, during an independent in-club exercise workout (FIG. 5), the user 8 scans the CMU 30 at the individual workstations (i.e. rower 106 or bike 102). The CMU 30 receives the uploaded machine and heart rate data from the applicable sensors 66c, 66a via the radio module 46.

The CMU 30 sends the user and CMU identification to the health club computer 120 which returns the information to the user 8 via the CMU 30 or a club display as feed back. Optionally the health club computer 120 may be used to upload heart rate and joule expenditure data directly to the CPU 20.

In an alternative club mode during group exercising, an instructor selects the desired in-club programme. The user's “login” is identified by a club RFID reader 122. The group programme and activity parameters are downloaded to the CMU 30 from the club computer 120 via the radio module 46. During the workout the CMU 30 collects data from the sensors 60, 62, 64, 66 and transmits it to the club computer 120. The club computer 120 may then both display the user performance on a projector array 126 as feedback, and upload the heart rate and energy joule data to the CPU 20.

The clock/micro-processor 52 allows the data collected by the heart rate sensor 60, accelerometer 62, GPS 64 and health club equipment sensors 66 to be time-stamped and compared against the caloric input values for the pre-selected meals. The microprocessor 52 preferably also controls all CMU 30 operational features, and includes self-testing and diagnostic functions. This information is uploadable to the CPU 20 so that functional errors with the CMU 30 can be indicated to the user 8. The microprocessor 52 manages the CMU 30 data storage/memory 56.

In another mode of operation the heart rate sensor 60 may be omitted and the CMU 30 and CPU 20 operated to extrapolate heart rate data and/or indications of the health of the user's 8 heart. In particular, the user's 8 lifestyle and/or biometric parameters and health activity level used to extrapolate heart activities required to achieve caloric expenditures and/or weight gain/loss over a selected time segment.

In the embodiment shown in FIG. 4, the CMU display 80 consists of a circular LED array which is divided into multiple sections. Although not essential, each section is preferably operable to provide feedback to the user 8 through a colour indication display. In a simplified design, the sections of the display 80 incorporate LEDs which are operable to emit the colours red, yellow or green as visual prompts to the user 8. The display colours provide a visual indication of whether or not the user's 8 heart rate, caloric and/or joule expenditure and overall physical activity or total energy (joule) expenditure meets or exceeds the pre-selected target standards for the pre-selected fitness goal and the user's 8 caloric input at each particular time segment or time of day. The display 80 provides a visual colour indicator of the comparison of the measured state of the user's heart rate, caloric and energy consumption, activity rate, and health against pre-defined programme goals for both the particular day, and particular time segment as determined by the clock/microprocessor 52. The colour indicator feedback is activated by the output switch array 82, with the goal for the user 8 to achieve green for each section. It is to be appreciated that in an alternate construction the display 80 could include a video display operable to output video images and/or graphics to the user 8.

The internal processor 52 is operable to compare both the heart data signals, as well as the caloric and joule input and expenditure data signals which are received from the accelerometer sensor 62, GPS 64 and any health club equipment sensors 66. The processor 52 then compares the user's 8 caloric input and/or the measured user heart rate and joule data with the heart rate and joule expenditure standards which are contained in the pre-selected fitness programme 45 stored in the memory 54 and provide validated user performance data 88. Although not essential, most preferably the comparison between the measured data and the stored standards is performed on a real-time basis by the processor 52 throughout the entire day, as the user 8 wears the device. Concurrently, the display 80 provides the user 8 with a visual indication of his/her compliance with the heart rate and expenditure standards. This advantageously allows the user 8 to implement minor, or if necessary, major adjustment to his or her physical activities either in real-time or following each time segment, to ensure continued compliance with the pre-selected fitness programme 45. In one possible mode, the CMU 30 may produce a data table used to control the feedback light. This data is uploadable from the CMU 30 to the CPU 20 during the time it is connected to a PC 40. For the user 8, the data table is based on their information in a unique user account 24 stored on the CPU 20 at the portal. Data may, for example, include the following Table 1.

TABLE 1
Date and Time
Degree of Movement
Movement Type
Heart Rate
HRV
Dynamic Energy
Activity Type

The total PI expected could be calculated using Basal Metabolic Rate and Activity level (or similar), with or without other values based on the specific fitness goals of the user 8 depending on the user's 8 personal profile. This value is then allocated to specific pre-selected daily periods, as for example, is shown in Table 2.

TABLE 2

| | | General PI | | Special or |
|---|---|---|---|---|
| Time | | to be | Physiotherapy | Additional |
| Segment | Time | Achieved | Targets | Activity Targets |
| 1 | 12 am-6 am | 200 | 100 | 0 |
| 2 | 6 am-8 am | 400 | 0 | 0 |
| 3 | 1 pm-3 pm | 300 | 100 | 200 |
| 4 | 5 pm-9 pm | 600 | 0 | 0 |

The present system preferably also allows the user 8 at the end of each segment to “top up” by performing additional caloric burning exercises so as to reach the total target caloric and/or joule burn and/or heart rate levels for a particular day. With the indication that the target heart and/or overall caloric and/or energy expenditure levels for a time segment has not yet been achieved, the user 8 may download from the CPU 30 supplemental exercise routines consisting of music, video and/or instructions. This downloaded routine is selected to enable the user 8 to complete the required level of necessary physical activity to achieve the target fitness level and target heart level, and having regard to the actual measured values for the particular day. Following at the end of each day, the user may then upload from the CMU 30 data representative of actual physical activities and heart rate data.

In a preferred mode, the user's physical activity is measured throughout the day and tracked by the CMU 30 using the sensor array 54. The collected data is converted to the universal Performance Index (PI) scale based on the user's 8 pre-input physical characteristics and meal/caloric choices. Feedback is customized to activity and energy expenditures throughout a day based on the user's 8 weight loss and fitness goals and correlated fitness programme which has been pre-selected. The portability and light weight of the CMU 30 allows it to be worn by the user 8 throughout his or her waking day to collect data relating to the user's 8 overall heart rate, and energy and/or caloric expenditure. The data collected is uploaded (via PC 40) to the CPU 20 as a validated user performance data 32 to provide feedback to the user's nutritionist or weight loss consultant. The uploaded performance data 32 provides an indication of whether the user's 8 caloric and/or joules expended as a result of the user's 8 physical activity equals or exceeds the target level, and provides an indication if whether the user 8 is adhering to the meal plan.

Although the preferred embodiment describes the CMU 30 as having a clock/processor 52 and memory 56 operable to store and validate measured user performance data 32 with the downloaded standards, the invention is not so limited. In a more economical construction, data storage and validation processing may be achieved by way of a user's home personal computer 40. The PC 40 may also be provided to facilitate date and firmware downloads between the CPU 20 and CMU 30. User performance information from the CMU 30 may also be stored directly on the PC 40, allowing the user 8 to monitor any changes in performance data and obtain feedback therefrom. In a more preferred construction, a firmware version is saved which is unique to the device ID for the CMU 30 at the CPU 20. Any firmware updates will be posted on the CPU 20 and downloaded as appropriate.

In another embodiment the CMU 30 operates, based on understanding the user's 8 body type, age, height, body mass index, and/or with other biometric variables stored in the CPU 40 to calculate and predetermine the amount of calories a user 8 has been consuming on a daily basis to arrive at their current body weight. The approach of calculating this “trending caloric intake” TCI, does not require the use of stored meal planning or food selection data which measures the number of calories an individual is consuming but rather, may be calculated from tabulated empirical data for a variety of like activities, and similar biometric profiles. This approach advantageously minimizes data entry requirements otherwise needed to establish an effective exercise for selected health goal.

The process of determining trending caloric intake may be achieved each time the user 8 enters their body weight in the system software. By factoring all of the user biometric variables, comparing the variables with predetermined standards therefore, and tracking historical physical activity performed by the user 8 while wearing the CMU 30, the CPU 40 is operable to estimate exercise benefits on the body.

In an alternative operational mode, the CMU 30 is operable to determine the health of one's individual heart without the use of dedicated heart measuring sensors. The CMU 30 and CPU 40 compares biometric data for a target user; and tracks accurately the user's 8 health activity over a period of time. By determining the exertion levels required and duration of time exercise/physical activity was sustained to yield an energy burn necessary to sustain such activity, it is possible to extrapolate the energy necessary (in particular musculoskeletal system) to sustain the identified activity, an energy burn would require the cardiovascular system to be able to support a predetermined level of exertion.

Although not essential, in a preferred construction a digital audio player 98 is preferably also provided within the CMU 30. The digital audio player 98 is controlled by a volume control 91 (FIG. 4) and connects to an ear phone jack 93, allowing music and/or audio instructions related to the pre-selected fitness programme to be downloaded from the CPU 20 for playback to the user 8.

Although the detailed description describes and illustrates various preferred embodiments, the invention is not so limited. Many modifications and variations will occur to persons skilled in the art. For a definition of the invention, reference may now be had to the appended claims.

1. A fitness monitoring and coaching system for ensuring a user's compliance with a preselected fitness program over a selected time period, the system comprising, a data management system including, a reference database for storing datafiles providing preferred target heart rate and joule expenditure standards for a plurality of exercise programs, said exercise programs correlated to a user's biometric parameter and a lifestyle profile parameter, said biometric parameters comprising at least one of said user's age and weight, a client database for storing input client data indicative of said user's biometric parameters and a lifestyle profile information for said user, a processing unit operable to output as said pre-selected fitness program a selected one of said exercising programs correlated to said input client data, a wearable monitoring device for communicating with said data management system and being operable to measure said user's caloric and/or joule expenditure over the selected time period, the monitoring device including, an internal clock, a display, memory for storing said pre-selected exercise program as downloaded user specific heart rate and joule expenditure standards over the selected time period, an output for outputting heart rate data signals indicative of said user's actual measured or estimated heart rate over the selected time, an accelerometer for monitoring and outputting joule data signals indicative of at least one of movement by said user; an internal processor for comparing said heart rate data signals and said joule data signals to said user specific heart rate and joule expenditure standards over said selected time period, and outputting a compliance output value which is dependent on the comparison, and wherein the wearable monitoring device is operable to provide on said display in substantially real time a visual representation of said compliance output value, suggestive of a modification to the user's heart rate and/or joule expenditure.

2. The monitoring system of claim 1, wherein said biometric parameters comprise said user's age, sex and weight, and said lifestyle profile information comprises at least one of a categorization of said user's job-type, a categorization of said user's overall fitness level, and a categorization of whether said user is a smoker or non-smoker.

3. The monitoring system of claim 1, wherein said lifestyle profile parameters include data selected from pre-selected classes of job categories, and pre-selected classes of leisure activity categories.

4. The monitoring system of claim 1, wherein said wearable monitoring device is a portable monitoring device operable to provide an aural representation of said output value data, and the selected time period comprises a period of at least eight hours.

5. The monitoring system of claim 1, wherein the output includes a heart rate monitoring sensor for monitoring and measuring signals indicative of said user's measured heart rate selected time period comprises the average waking times over at least a one week interval.

6. The monitoring system as claimed in claim 1, wherein the data management system further includes a caloric intake database for storing caloric information respecting a plurality of pre-proportioned food choices, the lifestyle profile information further including caloric input data representative of a plurality of pre-proportioned food choices pre-selected by said user for consumption over said selected time period.

7. Use of the monitoring system as claimed in claim 1, comprising, inputting said client data into said client database, downloading said pre-selected fitness program to said wearable monitoring device in response to said input client data, and with said user wearing said wearable monitoring device during said selected time period, actuating said internal processor to compare said heart rate signals and said joule data signals to said user specific heart rate and joule expenditure standards to generate said compliance output value, and
outputting to said user in substantially real time on said display an indication of whether the heart rate signals and said joule data signals meet or exceed the user specific heart rate and joule expenditure standards at that particular time.

8. Use of the monitoring system of claim 6 comprising, inputting said client data into said client database, downloading said pre-selected fitness program to said wearable monitoring device in response to said input client data, with said user wearing said wearable monitoring device, actuating said internal processor to compare said heart rate signals and joule data signals to said user specific heart rate and joule expenditure standards to generate said compliance output value, and modifying the pre-proportioned food choices pre-selected by said user for consumption in response to said compliance output.

9. Use of the monitoring system as claimed in claim 8, wherein the pre-proportioned food choices comprise pre-prepared meals, the data management system being operable to output delivery and/or shipping information respecting the food choices pre-selected be the user in response to both the input client data and the compliance output value.

10. A monitoring system for monitoring and validating compliance with a pre-selected fitness program by a user, the system including, a data management system including, a reference database for storing a plurality of datafiles providing preferred target heart rate and caloric and/or joule expenditure standards for a plurality of exercise and nutrition programs correlated to a selected target weight and fitness profile, and at least one pre-selected biometric parameter selected from a user's age, and weight, a client database for storing client data indicative of biometric parameters of said user, a processing unit operable to output as said pre-selected fitness program a selected one of said exercise and nutrition programs which correlates at least in part to at least one of said user's weight and fitness profile, and said pre-selected biometric parameters,
a wearable monitoring device for communicating with said data management system and being operable to measure said user's total daily caloric expenditure, the monitoring device including, an internal clock, memory for storing said selected wellness and rehabilitation physiotherapy program as time dependent user specific heart rate and caloric and/or joule expenditure standards for a pre-selected time period, a heart rate monitoring sensor for monitoring and outputting heart rate data signals indicative of said user's heart rate, and an accelerometer for monitoring and outputting caloric and/or joule data signals indicative of at least one of movement by said user and physical force on at least part of said user's body, a processor for receiving said heart rate data signals and said caloric and/or joule data signals as input values and validating said input values to said user specific heart rate and caloric and/or joule expenditure standards over said pre-selected time period, and outputting a compliance output data indicative of any difference between said input values and said user specific heart rate and caloric and/or joule expenditure standards, a display for providing said user with a visual representation of said compliance output data.

11. The monitoring system as claimed in claim 1, wherein the data management system further includes a caloric intake database for storing caloric information respecting a plurality of pre-proportioned food choices, the target weight and fitness profile further including caloric input data representative of a plurality of pre-proportioned food choices pre-selected by said user for consumption over said at least part of selected time period.

12. The monitoring system of claim 10, wherein said lifestyle profile includes data selected from pre-selected job categories, and pre-selected leisure activity categories.

13. The monitoring system of any claim 10, wherein said wearable monitoring device is a portable monitoring device operable to provide said visual representation of said output value data substantially in real time.

14. The monitoring system of claim 10, wherein the pre-selected time period comprises an average waking day.

15. Use of the system as claimed in claim 10, comprising, inputting said client data into said client database, downloading said selected program to said wearable device in response to said input data, and with said user wearing said wearable device during said selected time period, actuating said internal processor to compare said heart rate signals and said joule data signals to said user specific heart rate and joule expenditure standard to output said compliance output data, and modifying the user's food choices selected for consumption in response to the compliance output data.

16. Use of the system as claimed in claim 15, wherein said wearable monitoring device is operated to provide a visual indication to said user in response to an actual or potential change to the food choices selected for consumption.

17. A method of using a wearable monitoring device for validating a user's compliance with an exercise program, the wearable monitoring device including an output for outputting heart rate data signals indicative of said user's actual measured or estimated heart rate, and an accelerometer for monitoring and outputting caloric and/or joule data signals indicative of at least one of movement by said user and physical force on at least part of said user's body, the method comprising, providing a reference database storing a plurality of datafiles for preferred target heart rate and caloric and/or joule expenditure standards for a plurality of fitness programs, said fitness programs correlated to a pre-selected biometric parameters and target weight and/or fitness levels, said biometric parameters comprising at least said user's age and weight, providing a client database storing input client data indicative of a user's age and weight,
downloading to the wearable monitoring device as said exercise program a selected one of said fitness program correlated to the user's input client data, validating as input values said heart rate data signals and said caloric and/or joule data signals to said user specific heart rate and caloric and/or joule expenditure over a pre-selected time period, and outputting a compliance output data indicative of any difference between said input values and said user specific heart rate and caloric and/or joule expenditure.

18. The method of claim 17 further comprising displaying to said user a visual representation of said compliance output data, the visual representation being displayed to said user substantially in at least a bi-hourly frequency substantially throughout the user's waking day.

19. The method of claim 17, wherein said output for outputting heart rate data signals comprise a heart rate monitoring sensor, and said heart rate signals are indicative of said user's actual measured heart rate.
